(19) United States
(12) Patent Application Publication
(10) Pub. No.: US 2007/0213370 A1
(43) Pub. Date: Sep. 13, 2007

(54) 5-HTP Combination Therapy

(75) Inventors: Connie Sanchez Morillo, West Millford, NJ (US); Toni D. Wolinsky, Haworth, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 11/747,603

(22) Filed: May 11, 2007

Related U.S. Application Data
(63) Continuation-in-part of application No. 11/601,503, filed on Nov. 17, 2006.
(60) Provisional application No. 60/738,169, filed on Nov. 18, 2005.

(51) Int. Cl. A61K 31/445; A61K 31/405; A61K 31/343; A61K 31/137

(52) U.S. Cl. 514/317; 514/419; 514/438; 514/469; 514/649

(57) ABSTRACT

The present invention relates to combination therapies and pharmaceutical compositions comprising a combination of 5-hydroxytryptophan and a serotonin reuptake inhibitor. The present invention provides a pharmaceutical composition comprising (i) a serotonin reuptake inhibitor and (ii) 5-hydroxytrytophan. The present invention further provides a pharmaceutical composition comprising (i) 5-hydroxytryptophan in an amount ranging from about 1 mg to about 75 mg; and (ii) a serotonin reuptake inhibitor. The present invention also provides a pharmaceutical composition comprising (i) a subclinical dose of a serotonin reuptake inhibitor; and (ii) 5-hydroxytryptophan.

This application is a continuation-in-part of U.S. Ser. No. 11/601,503, filed Nov. 17, 2006 which claims the benefit of U.S. Provisional Application No. 60/738,169, filed Nov. 18, 2005, the contents of all of which are hereby incorporated by reference.

### FIELD OF INVENTION

The present invention relates to combination therapies and pharmaceutical compositions comprising a combination of 5-hydroxytryptophan and a serotonin reuptake inhibitor with improved efficacy.

### BACKGROUND OF THE INVENTION

5-hydroxytryptophan (5-HTP) is the direct precursor to serotonin (5-hydroxytryptamine; 5-HT). In vivo, 5-HTP is decarboxylated to produce 5-HT. 5-HT levels in the brain are dependent on levels of 5-HTP in the central nervous system (CNS). No transport molecules are necessary to transport 5-HTP across the blood-brain barrier. 5-HTP has been clinically shown to increase production of serotonin in the brain and therefore 5-HTP administration has been suggested as a treatment for patients with mild or moderate depression (for review, see Meyers, S., Altern Med Rev. 2000 February, 5(1):64-71; and Birdsall, T. C., Altern Med Rev. 1998 August; 3(4):271-80).

Serotonin reuptake inhibitors (SRIs) have become first choice therapeutics in the treatment of affective disorders, especially depression, because they are effective, well tolerated and have a favorable safety profile compared to the classic tricyclic antidepressants.

However, there is virtually no pharmaceutical treatment known that does not, apart from its benefits to patients, also carry some degree of risk of adverse side effects. 5-HTP monotherapy has been associated with gastrointestinal (nausea, vomiting, diarrhea) and psychopathological (acute anxiety state, hypomania) side effects in open studies with human patients (Zmilacher, K., Battegay, R. and Gastpar, M., Neuropsychobiology. 1988, 20(1):28-35; Gijsman, H. J., et al., J Clin Psychopharmacol. 2002 April, 22(2):183-9). 5-HTP administration has been implicated as a possible cause of Eosinophilia-Myalgia Syndrome (for review, see Das, Y. T., et al., Toxicol Lett. 2004 Apr. 15; 150(1):111-22.). One approach to managing these risks of side effects may be to lower the dose of 5-HTP.

With respect to SRIs, possible side effects to be balanced against the known benefits of SRIs and to be managed may include sexual dysfunction and sleep disturbances. Many patients experience delayed onset of a therapeutic effect during SRI monotherapy. Further clinical studies on depression and anxiety disorders indicate that more than 30% of patients treated with SRI monotherapy as a class are non-responsive.

Observations about the varying potentiation effects of different SRIs when administered with 5-HTP in various animal models have been noted. For example, Sanchez, C. and Hyttel, J., European Journal of Pharmacology (1994) 264:241-247 observed that a subeffective dose of L, 5-HTP greatly potentiated the antiaggressive effect of citalopram and paroxetine in an isolation-induced aggression mouse model.

C. Sanchez, European Journal of Pharmacology (2003) 464:155-158, also tested co-administration of L, 5-HTP with citalopram or escitalopram in an ultrasonic vocalization rat model for anxiety. In that model, in which ultrasonic vocalization is theorized to mimic panic anxiety in the rat, it was observed that the anxiolytic response to co-treatment of L, 5-HTP with citalopram was slightly attenuated and co-treatment of L, 5-HTP with escitalopram was markedly enhanced. Concomitant treatment with R-citalopram produced a significant increase of ultrasonic vocalization compared to controls.

Thus, patients may benefit from administration of a lower dose of 5-HTP. Patients may also benefit from administration of a lower dose of an SRI. Furthermore, patients that do not respond to SRIs may benefit from a combination therapy of an SRI and 5-HTP. Such combination therapy includes lower doses of either SRI or 5-HTP, yet may achieve greater efficacy or earlier onset of therapeutic effect than with SRI or 5-HTP monotherapy.

### SUMMARY OF THE INVENTION

An objective of the present invention is to provide a pharmaceutical composition comprising (i) 5-hydroxytryptophan in an amount ranging from about 1 mg to about 75 mg; and (ii) a serotonin reuptake inhibitor.

Another objective of the present invention also is to provide a pharmaceutical composition comprising (i) a subclinical dose of a serotonin reuptake inhibitor; and (ii) 5-hydroxytryptophan.

Another objective of the present invention is to provide a pharmaceutical composition comprising (i) a serotonin reuptake inhibitor and (ii) 5-hydroxytrytophan.

### BRIEF DESCRIPTION OF THE FIGURES

FIG. 1. Effect of escitalopram and fluoxetine alone and in combination with 5-HTP on extra-cellular 5-HT in frontal cortex in freely moving rats. 5-HTP (25 mg/kg, s.c.) administered to rats at 60 minutes following injection of escitalopram (0.5 mg/kg s.c.) (n=7) or fluoxetine (10 mg/kg, s.c.) (n=6).

### DETAILED DESCRIPTION OF THE INVENTION

The present invention relates to a pharmaceutical composition comprising 5-hydroxytryptophan and a serotonin reuptake inhibitor.

As used herein, “subclinical dose” shall mean a dose in an amount less than the lowest dose that is approved as a monotherapy for marketing by a governmental regulatory agency as of the priority filing date of this application.

As used herein, “allosteric modulator” shall mean an SRI that has a Z-factor of greater than 0 (zero), which shall be determined by the method described herein.

As used herein, “serotonin dual action compound” shall mean a compound that both 1) binds to the primary binding site of the serotonin transporter having an IC₅₀ value of less than about 50 nM, and 2) binds to an allosteric site of the serotonin transporter having a Z-factor greater than zero (0), as determined by the methods described herein. In a further embodiment, the serotonin dual action compound further binds to the primary binding site of the serotonin transporter having an IC₅₀ value of less than about 10 nM.

5-hydroxytryptophan (5-HTP) is an aromatic amino acid naturally produced in the body from amino acid L-tryptophan. 5-HTP is the direct precursor to 5-HT. The formula of 5-HTP is shown below as Formula I.

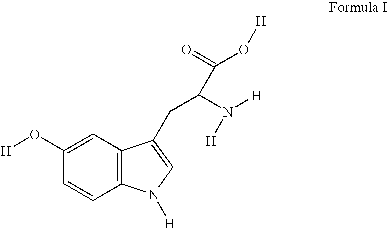

5-HTP is also known as 2-amino-3-(5-hydroxy-1H-indol-3-yl)-propanoic acid (C₁₁H₁₂N₂O₃). Throughout the description and the claims, “5-HTP” and “5-hydroxytrytophan” are intended to include any form of the amino acid 5-hydroxytryptophan, including the base (zwitter ion), pharmaceutically acceptable salts, hydrates or solvates of the base or salt, as well as anhydrates, and also amorphous, or crystalline forms. As used herein, “pharmaceutically acceptable salts” includes salts with pharmaceutically acceptable acids or bases. With respect to 5-HTP, such salts may be formed with pharmaceutically acceptable bases, particularly strong bases such as sodium potassium or ammonium hydroxide. Such salts of 5-HTP may also be formed with pharmaceutically acceptable acids, such as hydrochloric acid, hydrobromic acid, phosphoric acid, maleic acid, oxalic acid, tartaric acid and the like. Accordingly, 5-HTP may be used in the form of an acid addition salt, or in the form of a zwitter ion hydrate, zwitter ion monohydrate, or zwitter ion anhydrate.

For the purposes of this invention, 5-HTP may be in a racemic mixture or as the substantially pure D-enantiomer, D-5-hydroxytryptophan, or as the substantially pure L-enantiomer, L-5-hydroxytryptophan.

One aspect of the present invention relates to a pharmaceutical composition comprising 5-HTP for use in a combination therapy with an SRI.

Another aspect of the present invention provides a pharmaceutical composition comprising (i) 5-hydroxytryptophan in an amount ranging from about 1 mg to about 75 mg; and (ii) a serotonin reuptake inhibitor.

In accordance with the present invention described herein, 5-HTP may be used to augment and/or provide an earlier onset of the therapeutic effect of serotonin reuptake inhibitors. Further, as part of the present invention, lower doses of 5-HTP when used in combination therapy may augment and/or provide an earlier onset of the therapeutic effect of an SRI. In one embodiment of the invention, 5-HTP in an amount ranging from about 1 mg to about 75 mg is coadministered with an SRI. In another embodiment of the invention, 5-HTP in an amount ranging from about 3 mg to about 50 mg is coadministered with an SRI. In still another embodiment of the invention, 5-HTP in an amount ranging from about 10 mg to about 50 mg is coadministered with an SRI.

As used herein, augmenting shall mean improving the therapeutic effect and/or potentiating the effect of an SRI.

Many compounds with serotonin reuptake inhibiting effect have been described in the literature. Any pharmacologically active compound, which primarily or partly exerts its therapeutic effect by binding to the primary ligand binding site of the serotonin transporter to inhibit serotonin reuptake in the central nervous system (CNS), may benefit from augmentation with 5-HTP.

The following list contains a number of serotonin reuptake inhibitors which may benefit from augmentation or combined administration with 5-HTP: citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, O1 77, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine, desmethylsubitramine, didesmethylsubitramine, clovoxamine vilazodone, N-[(1-[(6-Fluoro-2-napthalenyl)methyl]-4-piperidinyl]amino]carbonyl]-3-pyridine carboxamide, [trans-6-(2-chlorophenyl)-1,2,3,5,6,10b-hexahydropyrrolo-(2,1-a)isoquinoline] (McN 5707), (dl-4-exo-amino-8-chloro-benzo-(b)-bicyclo [3.3.1] nona-2-6 alpha (10 alpha)-diene hydrochloride) (Org 6997), (dl)-(5 alpha,8 alpha,9 alpha)-5,8,9,10-Tetrahydro-5,9-methanobenzocycloocten-8-amine hydrochloride (Org 6906), -[2-[4[(6-fluoro-1H-indol-3-yl)-3,6-dihydro-1(2H)-pyridinyl]ethyl]-3-isopropyl-6-(methylsulphonyl)-3,4-dihydro-1H-2,1,3-benzothiadiazine-2,2-dioxide (LY393558), [4-(5,6-dimethyl-2-benzofuranyl)-piperidine] (CGP 6085), dimethyl-[5-(4-nitro-phenoxy)-6,7,8,9-tetrahydro-5H-benzocyclohepten-7-yl]amine (RU 25.591),

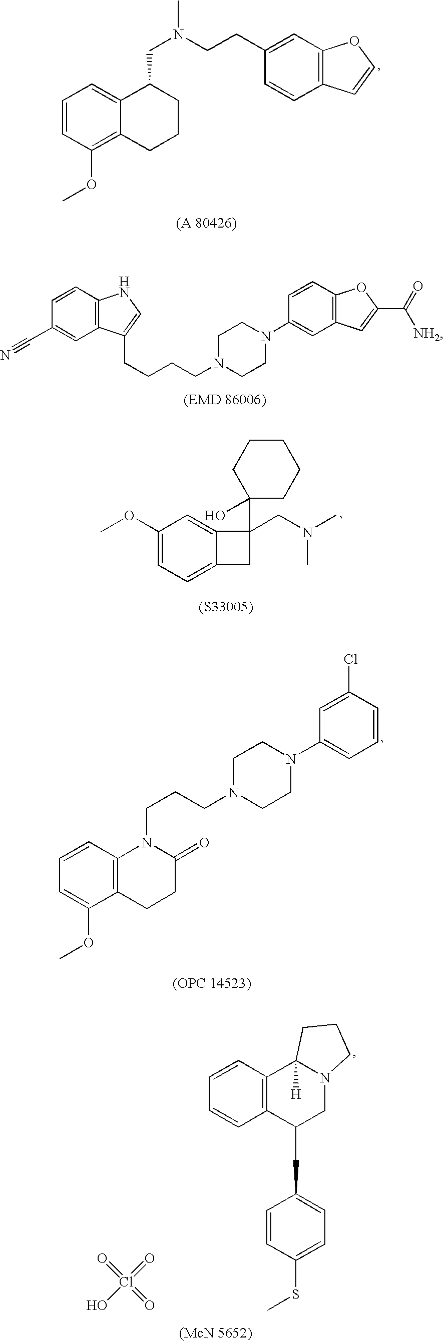

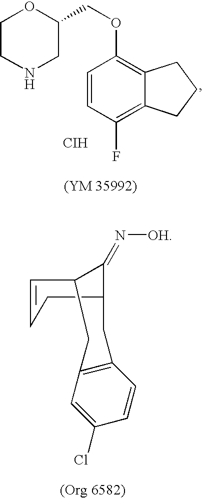

In one embodiment, the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

As used herein, the term selective serotonin reuptake inhibitor (SSRI) means an inhibitor of monoamine transporters, which has a stronger inhibitory effect at the serotonin transporter (SERT) than the norepinephrine transporter as measured by in vitro reuptake inhibitory potency.

SSRIs are to be considered as part of the class serotonin reuptake inhibitors and therefore may be used according to the present invention. Thus, in a further embodiment, the SRI may be an SSRI such as citalopram, escitalopram, fluoxetine, fluvoxamine, sertraline, or paroxetine.

The compounds mentioned above may be used in the form of the racemate or the pure enantiomer. In some embodiments of the present invention, citalopram is in the form of the racemate, or racemic mixture of the R-(−)-enantiomer (R-citalopram) and the S-(+)-enantiomer (S-citalopram).

In another embodiment, S-citalopram, also known as escitalopram, is the pure enantiomer. The IUPAC name for escitalopram is S-(+)-1-[3-(dimethylamino)propyl]-1-(p-fluorophenyl)-5-phthalancarbonitrileoxalate.

In another embodiment, paroxetine is the pure enantiomer. The IUPAC name for paroxetine is (3S,4R)-3-((1,3-benzodioxol-5-yloxy)methyl)-4-(4-fluorophenyl)-piperidine.

The compounds mentioned above may be used in the form of the free base or in the form of a pharmaceutically acceptable salt, such as an acid addition salt, the latter being obtainable by a reaction of the base form with an appropriate acid. Each of the serotonin reuptake inhibitors specified above is intended to be an individual embodiment. Accordingly, each compound may be claimed individually.

For example, citalopram may be used in the form of the hydrobromide or the free base; escitalopram in the form of the oxalate, hydrobromide, or the free base; fluoxetine and sertraline in the form of the hydrochloride; paroxetine in the form of the hydrochloride or the mesylate; and fluvoxamine in the form of the maleate.

In another embodiment of the invention, the SRI may be an allosteric modulator.

As used herein, “allosteric modulator” shall mean an SRI that has a Z-factor of greater than 0 (zero), which shall be determined by the method described herein.

In a further embodiment, the allosteric modulator may be selected from escitalopram and paroxetine.

In still further embodiments, the SRI may be a serotonin dual action compound.

As defined herein, a “serotonin dual action compound” shall mean a compound that both 1) binds to the primary binding site of the serotonin transporter having an IC₅₀ value of less than about 50 nM, and 2) binds to an allosteric site of the serotonin transporter having a Z-factor greater than zero (0), as determined by the methods described herein.

In a further embodiment, the serotonin dual action compound further binds to the primary binding site of the serotonin transporter having an IC₅₀ value of less than about 10 nM.

In another embodiment, the serotonin dual action compound may be selected from escitalopram and paroxetine.

As mentioned above, the combination of 5-HTP with an SRI unexpectedly shows a synergistic effect on the central nervous system. Thus in one embodiment of the invention, lower doses of 5-HTP than normally used in monotherapy may be used in combination with a dose of serotonin reuptake inhibitor normally used in monotherapy to augment the 5-HT output and thereby may provide an earlier onset of the therapeutic effect of serotonin reuptake inhibitors.

In some embodiments, the amount of 5-HTP to be used in combination therapy may range from about 1 to about 75 mg per day, such as from about 3 to about 50 mg per day, or from about 10 to about 50 mg per day. Pharmaceutical compositions of the present invention may therefore comprise from about 1 to about 75 mg, such as from about 3 to about 50 mg, or from about 10 to about 50 mg 5-HTP.

Serotonin reuptake inhibitors, and serotonin dual action compounds, including the SSRIs and allosteric modulators specifically mentioned hereinabove, differ both in molecular weight and in activity. As a consequence, the amount of serotonin reuptake inhibitor or serotonin dual action compound used in combination therapy depends on the nature of said serotonin reuptake inhibitor. In one embodiment, the serotonin reuptake inhibitor, serotonin dual action compound, SSRI or allosteric modulator, is administered in a therapeutically effective amount.

In another embodiment of the invention, the pharmaceutical composition contains a therapeutically effective amount of escitalopram. In a further embodiment of the invention, the pharmaceutical composition contains from 5 mg to 30 mg of escitalopram. Also included in the present invention is the administration of such pharmaceutical compositions to a patient in need thereof, so that the daily dose ranges of escitalopram are 5 mg to 30 mg per day.

In another embodiment of the invention, the pharmaceutical composition contains a therapeutically effective amount of paroxetine. In a further embodiment of the invention, the pharmaceutical composition contains from 10 mg to 60 mg of paroxetine. Also included in the present invention is the administration of such pharmaceutical compositions to a patient in need thereof, so that the daily dose ranges of paroxetine are 10 mg to 60 mg per day.

In accordance with the present invention, combination therapy using 5-HTP with a subclinical dose of an SRI normally used in monotherapy may have the advantage that a beneficial central nervous system effect may be obtained in the large number of patients that do not respond to conventional monotherapy with SRIs.

It has unexpectedly been shown that subclinical doses of an SRI may be used in combination with 5-HTP to augment and/or provide an earlier onset of the therapeutic effect of the SRI.

In a further aspect of the invention, combination therapy using 5-HTP with a subclinical dose of serotonin reuptake inhibitor, may be used to augment the therapeutic effect and/or to reduce side-effects associated with larger amounts of SRI used in monotherapy.

Accordingly, one aspect of the present invention relates to a pharmaceutical composition comprising (i) a subclinical dose of a serotonin reuptake inhibitor; and (ii) 5-hydroxytryptophan.

As used herein, “subclinical dose” shall mean a dose in an amount less than the lowest dose that is approved as a monotherapy for marketing by a governmental regulatory agency as of the priority filing date of this patent application.

In further embodiments of the invention, the amount of 5-HTP to be used in combination therapy may range from about 1 mg to about 600 mg per day, such as from about 25 mg to about 300 mg per day, or from about 50 mg to about 200 mg per day. Pharmaceutical compositions of the present invention may therefore comprise from about 1 mg to about 600 mg, such as from about 25 mg to about 300 mg, or from about 50 mg to about 200 mg 5-HTP.

In another embodiment, the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

In a further embodiment, the SRI may be an SSRI, such as citalopram, escitalopram, fluoxetine, fluvoxamine, sertraline, or paroxetine.

In another embodiment of the invention, the SRI may be an allosteric modulator.

In a further embodiment, the allosteric modulator may be selected from escitalopram and paroxetine.

In other embodiments, the SRI may be a serotonin dual action compound. In still other embodiments, the serotonin dual action compound may be selected from escitalopram and paroxetine.

Accordingly, one embodiment of the present invention includes a pharmaceutical composition comprising a subclinical dose of an allosteric modulator and 5-HTP, wherein the composition comprises 5-HTP in an amount ranging from about 1 mg to about 600 mg, from about 25 mg to about 300 mg, or from about 50 mg to about 200 mg. Another embodiment includes a pharmaceutical composition comprising a subclinical dose of a serotonin dual action compound and 5-HTP, wherein the composition comprises 5-HTP in an amount ranging from about 1 mg to about 600 mg, from about 25 mg to about 300 mg, or from about 50 mg to about 200 mg. Also included in the present invention is the administration of such pharmaceutical compositions to a patient in need thereof, so that the daily dose ranges are from about 1 mg to about 600 mg per day, or about 25 mg to about 300 mg per day, or about 50 mg to 200 mg per day

In one aspect of the invention, the pharmaceutical composition comprises a subclinical dose of escitalopram. For example, the pharmaceutical composition may comprise escitalopram in an amount less than 5 mg.

In yet another aspect of the invention, the pharmaceutical composition comprises escitalopram in an amount less than 5 mg and 5-HTP in an amount ranging from about 1 mg to about 600 mg. In another embodiment of the invention, the pharmaceutical composition comprises escitalopram in an amount less than 5 mg and 5-HTP in an amount ranging from about 25 mg to about 300 mg. In still another embodiment of the invention, the pharmaceutical composition comprises escitalopram in an amount less than 5 mg and 5-HTP in an amount ranging from about 50 mg to about 200 mg.

In a further embodiment of the invention, the pharmaceutical composition comprises escitalopram in an amount from about 0.1 mg to about 4.9 mg. In another embodiment, the pharmaceutical composition comprises escitalopram in an amount from about 0.5 mg to about 4.5 mg. In still another embodiment, the pharmaceutical composition comprises escitalopram in an amount from about 1 mg to about 4 mg.

In one aspect of the invention, the pharmaceutical composition comprises a subclinical dose of paroxetine. In one aspect of the invention, the pharmaceutical composition comprises paroxetine in an amount less than 10 mg.

In another aspect of the invention, the pharmaceutical composition comprises paroxetine in an amount less than 10 mg and 5-HTP in an amount ranging from about 1 mg to about 600 mg. In another embodiment of the invention, the pharmaceutical composition comprises paroxetine in an amount less than 10 mg and 5-HTP in an amount ranging from about 25 mg to about 300 mg. In still another embodiment of the invention, the pharmaceutical composition comprises paroxetine in an amount less than 10 mg and 5-HTP in an amount ranging from about 50 mg to about 200 mg.

In a further embodiment of the invention, the pharmaceutical composition comprises paroxetine in an amount from about 0.1 mg to about 9.9 mg. In another embodiment, the pharmaceutical composition comprises paroxetine in an amount from about 0.5 mg to about 9.5 mg. In still another embodiment, the pharmaceutical composition comprises paroxetine in an amount from about 1 mg to about 9 mg.

Another aspect of the present invention relates to a pharmaceutical composition comprising (i) a serotonin reuptake inhibitor; and (ii) 5-hydroxytryptophan.

In further embodiments of the invention, the amount of 5-HTP to be used in combination therapy may range from about 1 mg to about 600 mg per day, such as from about 25 mg to about 300 mg per day, or from about 50 mg to about 200 mg per day. Pharmaceutical compositions of the present invention may therefore comprise from about 1 mg to about 600 mg, such as from about 25 mg to about 300 mg, or from about 50 mg to about 200 mg 5-HTP.

In another embodiment, the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

In a further embodiment, the SRI may be an SSRI, such as citalopram, escitalopram, fluoxetine, fluvoxamine, sertraline, or paroxetine.

In another embodiment of the invention, the SRI may be an allosteric modulator.

In a further embodiment, the allosteric modulator may be selected from escitalopram and paroxetine.

In other embodiments, the SRI may be a serotonin dual action compound. In still other embodiments, the serotonin dual action compound may be selected from escitalopram and paroxetine.

Accordingly, one embodiment of the present invention includes a pharmaceutical composition comprising an allosteric modulator and 5-HTP, wherein the composition comprises 5-HTP in an amount ranging from about 1 mg to about 600 mg, from about 25 mg to about 300 mg, or from about 50 mg to about 200 mg. Also included in the present invention is the administration of such pharmaceutical compositions to a patient in need thereof, so that the daily dose ranges are from about 1 mg to about 600 mg per day, or about 25 mg to about 300 mg per day, or about 50 mg to 200 mg per day

In one aspect of the invention, the pharmaceutical composition comprises escitalopram. For example, the pharmaceutical composition may comprise escitalopram in an amount ranging from about 5 mg to about 30 mg.

In yet another aspect of the invention, the pharmaceutical composition comprises escitalopram in an amount from about 5 mg to about 30 mg and 5-HTP in an amount ranging from about 1 mg to about 600 mg. In another embodiment of the invention, the pharmaceutical composition comprises escitalopram in an amount from about 5 mg to about 30 mg and 5-HTP in an amount ranging from about 25 mg to about 300 mg. In still another embodiment of the invention, the pharmaceutical composition comprises escitalopram in an amount ranging from about 5 mg to about 30 mg and 5-HTP in an amount ranging from about 50 mg to about 200 mg.

In one aspect of the invention, the pharmaceutical composition comprises paroxetine. In one aspect of the invention, the pharmaceutical composition comprises paroxetine in an amount ranging from about 10 mg to about 60 mg.

In another aspect of the invention, the pharmaceutical composition comprises paroxetine in an amount from about 10 mg to about 60 mg and 5-HTP in an amount ranging from about 1 mg to about 600 mg. In another embodiment of the invention, the pharmaceutical composition comprises paroxetine in an amount from about 10 mg to about 60 mg and 5-HTP in an amount ranging from about 25 mg to about 300 mg. In still another embodiment of the invention, the pharmaceutical composition comprises paroxetine in an from about 10 mg to about 60 mg and 5-HTP in an amount ranging from about 50 mg to about 200 mg.

Aromatic amino acid decarboxylases that degrade 5-HTP to serotonin are widely distributed throughout the body. A peripheral decarboxylation inhibitor can be administered in combination with 5-HTP to prevent the degradation of 5-HTP to serotonin.

Thus, the pharmaceutical composition may further comprise a peripheral decarboxylation inhibitor. Peripheral decarboxylation inhibitors include, but are not limited to, carbidopa, L-α-methyldopa, monofluoromethyldopa, difluoromethyldopa and benserazide.

Pharmaceutical compositions of the present invention may contain carbidopa in an amount ranging from about 100 mg to about 150 mg.

According to the invention, the pharmaceutical compositions described herein may be administered in any suitable way, e.g. orally or parentally, and it may be presented in any suitable form for such administration, e.g. in the form of tablets, capsules, powders, syrups or solutions or dispersions for injection. In one embodiment of the present invention, the composition is administered in the form of a solid pharmaceutical entity, suitably as a tablet or a capsule or in the form of a suspension, solution or dispersion for injection.

Methods for the preparation of solid pharmaceutical compositions are well known in the art. For example, tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatin, gums, and the like. Other adjuvants or additives such as colorings, aroma, preservatives, etc. may also be used provided that they are compatible with the active ingredients.

The pharmaceutical compositions can be administered as part of the claimed invention as an oral dose form, such as a solid dose form, typically tablets or capsules, or as a liquid oral dose form. The pharmaceutical compositions described herein are most conveniently administered in unit dosage forms such as tablets or capsules. For example, such tablets or capsules may contain 5-HTP in amounts ranging from about 1 to about 600 mg, or from about 25 mg to about 300 mg, or from about 10 to 50 mg.

To prepare the pharmaceutical composition of this invention, an appropriate amount of 5-HTP and/or serotonin reuptake inhibitor, in salt form or base form, is combined in an intimate admixture with a pharmaceutically acceptable carrier, which can take a wide variety of forms depending on the form desired for administration. Those pharmaceutical compositions may be in unitary dosage form suitable for administration orally, rectally, percutaneously, or by parenteral injection. For example, in preparing the compositions in oral dosage form, any of the usual pharmaceutical media, such as water, glycols, oils, alcohols, and the like, may be incorporated in the form of oral liquid preparations. Oral liquid preparations may be suspensions, syrups, elixirs, and solutions. In preparing the compositions in oral dosage form, any of the usual pharmaceutical media, such as starches, sugars, kaolin, lubricants, binders, disintegrating agents, and the like, may be incorporated in the form of solid carriers. Oral solid preparations may be powders, pills, capsules and tablets. Because of their ease in administration, tablets and capsules represent the most advantageous oral dosage form, in which case solid pharmaceutical carriers would be employed.

It is especially advantageous to formulate the aforementioned pharmaceutical compositions in a unitary dosage form for ease of administration and uniformity of dosage. As used herein, unitary dosage form means physically discrete units suitable as unitary dosages, each unit containing a predetermined quantity of 5-HTP and/or serotonin reuptake inhibitor calculated to produce the desired therapeutic effect, in association with the required pharmaceutical carrier. Examples of unitary dosage forms are tablets (including scored coated tablets), capsules, pills, powder packets, wafers, injectable solutions or suspensions, and the like, and combinations thereof.

5-HTP may be administered before, during or after the administration of the SRI provided that the time between administration of 5-HTP and the administration of the SRI is such that ingredients are allowed to act synergistically on the central nervous system. When simultaneous administration of 5-HTP and an SRI is envisaged, a single composition containing both an SRI and 5-HTP may be particularly convenient. Alternatively, the serotonin reuptake inhibitor and 5-HTP may be administered separately in the form of suitable compositions. Such pharmaceutical compositions may further comprise a peripheral decarboxylation inhibitor. The compositions may be prepared as described hereinabove. Thus, such compositions may comprise an SRI and a peripheral decarboxylation inhibitor, such as carbidopa. Other compositions may comprise 5-HTP and a peripheral decarboxylation inhibitor, such as carbidopa. Such compositions may be administered simultaneously, such as in a single tablet, and the like, or may be administered separately, such as in separate compositions or tablets, and the like.

The present invention also comprises 5-HTP and an SRI as a combination preparation for simultaneous, separate or sequential use in psychiatric drug therapy. Such compositions may comprise, for example, a kit comprising discrete unit dosage forms containing 5-HTP and discrete unit dosage forms of an SRI, all contained in the same container or pack, e.g. a blister pack. Such pharmaceutical compositions may further comprise a peripheral decarboxylation inhibitor. The above mentioned compositions are made in accord with any aspects of the present invention described herein.

In some embodiments, the invention relates to a kit comprising a subclinical dose of an SRI and 5-HTP. In some embodiments, the invention relates to a kit comprising a subclinical dose of serotonin reuptake inhibitor and 5-HTP in an amount ranging from about 1 mg to about 600 mg, in an amount ranging from about 25 mg to about 300 mg or in an amount ranging from about 50 mg to about 200 mg.

In some embodiments, the invention relates to a kit comprising an SRI and 5-HTP in an amount ranging from about 1 mg to about 75 mg, in an amount ranging from about 3 mg to about 50 mg or in an amount ranging from about 10 mg to about 50 mg.

In other embodiments, the invention relates to a kit comprising an SRI and 5-HTP. In some embodiments, the invention relates to a kit comprising an SRI and 5-HTP in an amount ranging from about 1 mg to about 600 mg, in an amount ranging from about 25 mg to about 300 mg or in an amount ranging from about 50 mg to about 200 mg.

In other aspects, the invention relates to the pharmaceutical compositions as described herein comprising 5-HTP and an SRI for use in combination therapy for the treatment of affective disorders. In another aspect of the invention, the invention relates to the pharmaceutical compositions as described herein comprising 5-HTP and an SRI for use in combination therapy for the treatment of depression. In still another aspect, the present invention relates to the pharmaceutical compositions as described herein comprising 5-HTP and an SRI for use in combination therapy for the treatment of anxiety disorders.

Such pharmaceutical compositions may further comprise a peripheral decarboxylation inhibitor.

In other aspects, the invention relates to the use of 5-HTP for the preparation of a pharmaceutical composition to be used in combination with an SRI. In a further aspect, the invention relates to the use of 5-HTP for the preparation of a pharmaceutical composition useful for augmenting and/or providing an earlier onset of the therapeutic effect of an SRI.

In still further aspects, the invention relates to a method of treatment of diseases or disorders responsive to an SRI, comprising administering 5-HTP and an SRI to a human patient in need thereof.

A further aspect of the invention relates to use of 5-HTP and an SRI for the preparation of a pharmaceutical composition for the treatment of diseases or disorders responsive to the therapeutic effect of an SRI.

In another aspect, the invention relates to use of 5-HTP for the preparation of a pharmaceutical composition for the treatment of an individual to be treated with or undergoing treatment with an SRI, wherein said individual suffers from diseases or disorders responsive to the therapeutic effect of an SRI. In some aspects, the invention relates to use of 5-HTP for the preparation of a kit for the treatment of an individual to be treated with or undergoing treatment with an SRI, wherein said individual suffers from diseases or disorders responsive to the therapeutic effect of an SRI.

In other embodiments, the invention relates to a method for augmenting and/or providing an earlier onset of the therapeutic effect of an SRI comprising administering 5-HTP to a human patient to be treated with or undergoing treatment with an SRI.

In another embodiment, the pharmaceutical compositions as described herein are used in the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder, and drug abuse, in particular depression.

In further embodiments, the pharmaceutical compositions as described herein are used in the treatment of anxiety disorders includes general anxiety disorder, panic anxiety, obsessive compulsive disorder, acute stress disorder, post trauma stress disorder, or social anxiety disorder.

Experimental Details

Mouse Forced Swim Test

Male NMRI/BOM mice (18-25 g; Bomholtgaard, Denmark) were used. The mice were housed in plastic cages (35×30×12 cm), 10 in each and habituated to the animal facilities for at least a week before test. The room temperature (21+/−2° C.), relative humidity (55+/−5%), and air exchange (16 times per h) were automatically controlled. The animals had free access to commercial food pellets and tap water before test.

A mouse that is forced to swim in a spatially constrained container will exert a characteristic immobile posture. Pretreatment with an antidepressant will counteract this effect. The test was conducted as described in detail by Sanchez and Meier (Psychopharmacol. 129: 197-205; 1997). Briefly, a fully automated test system with 6 swim units (2000 ml glass jars filled with 1200 ml soiled water (23-25° C.) in which a mouse had been placed previously) was used. The assessment of immobility was performed by image analysis.

Thirty minutes after drug or vehicle treatment the mice were treated with 5-HTP and 20 min later the mice were placed into the glass jar and left in the water for a total of 6 min. The accumulated duration of immobility was measured during the last 3 min. A total of 9-18 mice were tested per dose.

Rat Microdialysis

Microdialysis in freely moving rats was performed as described in detail by Mark, A., Kreilgaard, M. and Sanchez, C. (Neuropharmacology. 2003 August, 45(2):167-73) to study the effect of escitalopram and fluoxetine alone, and in combination with 5-HTP (25 mg/kg, s.c.) on extracellular serotonin levels.

Briefly, male Sprague-Dawley rats were prepared for microdialysis by surgically implanting intracerebral guide cannulas. A microdialysis probe was inserted through the guide cannula. Perfusion of the microdialysis probe with filtered Ringer solution (146 mM NaCl, 3 mM KCl, 1 mM MgCl₂, 1.2 mM CaCl₂) was done before the insertion of the probe and continued for the duration of the experiments at a constant flow of 1 μl/minute into the frontal cortex. After stabilization of the animals, testing was initiated by the injection of test compound (escitalopram 0.5 mg/kg, s.c. or fluoxetine 10 mg/kg s.c.). A 20 minute sampling regime was used throughout the experiment. 5-HTP (25 mg/kg, s.c.) was injected 60 minutes following injection of test compound. 5-HT levels in the dialysate were measured in each sample by means of HPLC with electrochemical detection.

Mouse Marble Burying Behavior

Male BALB/cByJ mice (Jackson labs, Bar Harbor, Me.) were housed 5/cage upon arrival, at which time they were 7-8 weeks of age. Animals acclimated to the housing facility under standard laboratory conditions for a period of at least one week before testing (lights on at 6:00 AM). Following a one hour period of acclimation to the test room, animals were dosed with either vehicle (saline) or escitalopram (0.0625, 0.125, or 0.25 mg/kg, i.p.) Thirty minutes later, animals received an injection of vehicle or 5-HTP (2.5 mg/kg, i.p.). Fifteen minutes after the second injection, animals were individually placed into novel cages in which a layer of Aspen Pine bedding on which two parallel rows of 10 marbles each (i.e. twenty total) were placed. After 30 minutes had elapsed, the mice were removed from their test cages and returned to their home cages. The number of fully visible marbles (less than ⅔ covered with bedding) were counted and subtracted from 20 to arrive at the number of marbles buried.

Inhibition of ³H-Serotonin Uptake in Rat Brain Synaptosomes

In order to test compounds at the primary, high-affinity binding site of the serotonin transporter, i.e. determine if a compound is a serotonin reuptake inhibitor, inhibition of the uptake of serotonin (5-HT) is determined.

By the following method, the inhibition of the uptake of ³H-serotonin (3H-5-HT) (10 nM) in rat brain synaptosomes by test compounds is determined in vitro. The method, and also results of serotonin uptake for specific SRIs, is described in Hyttel, J., Psychopharmacology 1978, 60:13-18; Hyttel, J., Prog. Neuro-Psychopharmacol. & Biol. Psychait. 1982, 6:277-295; Hyttel, J. & Larsen, Acta Pharmacol. Tox. 1985, 56(suppl. 1):146-153; Sanchez, C. and Hyttel, J. European J. Pharm. 1994, 264:241-247; and Bøgesø, K., et al, U.S. Pat. No. 4,943,590, issued Jul. 24, 1990.

Procedure: Male Wistar (Mol:Wist) rats (125-250 g) are sacrificed by decapitation and exanguinated. Brain tissue (minus cerebellum) is gently homogenized (glass Teflon homogenizer) in 40 vol (w/v) of ice-cold 0.32M of sucrose containing 1 mM of nialamide. The P2 fraction (synaptosomal fraction) is obtained by centrifugation (600×g, 10 min and 25000×g, 55 min, 4° C.) and suspended in 800 volumes of a modified Krebs-Ringer-phosphate buffer, pH 7.4.

To 400 μl of the synaptosomal suspension (5 mg original tissue) on ice, 100 μl test compound in water is added. After preincubation at 37° C. for 5 min, 100 μl of ³H-5-HT (final concentration 10 nM) is added and the samples are incubated for 10 min at 37° C. The incubation is terminated by filtering the samples under vacuum through Whatman GF/F filters with a wash of 5 ml buffer containing 10 μM of unlabelled 5-HT. The filters are placed in counting vials and 4 ml of appropriate scintillation fluid (e.g. Picofluor™l5, Packard) is added. After shaking for 1 h and storage for 2 h in the dark, the content of radioactivity is determined by liquid scintillation counting (cpm). Uptake is obtained by subtracting the non-specific binding and passive transport measured in the presence of 10 μM test compound. The measured cpm is plotted against test compound concentration, and the best fitting s-shaped curve is drawn. The uptake inhibitory potencies are expressed as IC₅₀ values in nM (logarithmic means). Two full concentration-response curves can be measured using five concentrations of test compound in triplicate. The IC₅₀ value is determined as the concentration at which the uptake is 50% of the total uptake in control samples minus the non-specific binding and uptake in the presence of 10 μM test compound.

Thus, as used in the specification and claims, a serotonin reuptake inhibitor (SRI) that binds with high affinity at the serotonin transporter primary binding site, i.e. has stronger uptake inhibitory potency, is determined by the above test as a compound having an IC₅₀ value less than about 50 nM.

In still further embodiments, SRIs that bind to the primary binding site of the serotonin transporter having an IC₅₀ value of less than about 10 nM have even stronger uptake inhibitory potency.

Allosteric Modulation of the Serotonin Transporter

The allosteric site of a protein is an additional binding site, which is distinct from the primary ligand binding site. Compounds that modulate, for instance increase and/or stabilize, binding between the ligand and the ligand binding site are generally considered to operate through an allosteric mechanism.

While not wishing to be bound by a particular theory, the serotonin transporter is considered to have at least two separate binding sites: a primary, high-affinity binding site that mediates the inhibition of serotonin reuptake, and one or more low-affinity binding sites that allosterically modulate the binding of ligands at the primary site (Plenge, P., and Mellerup, E. T. Eur J Pharmacol. 1985 Dec. 10; 119(1-2):1-8; Wennogle, L. P. and Meyerson, L. R. Life Sci. 1985 Apr. 22; 36(16):1541-50).

The binding of escitalopram to an allosteric binding site on the SERT has been demonstrated in several studies. Studies of the interaction of escitalopram with the human serotonin transporter expressed in COS-1 cell membranes demonstrated that escitalopram binds to a secondary low-affinity allosteric site and retards the dissociation rate of ³H-escitalopram (used in a concentration that exclusively binds to the high-affinity primary site) from the transporter; that is, escitalopram appears to have a stabilizing/self-potentiating effect on the escitalopram:serotonin transporter complex. The effect of escitalopram is concentration-dependent (Chen, F., et al., Eur Neuropsychopharmacol. 2005 March; 15(2):193-8).

In addition to escitalopram, the interaction of paroxetine, sertraline, fluoxetine, venlafaxine, duloxetine, and serotonin with high- and low-affinity binding sites on the human serotonin transporter expressed in COS-1 cell membranes has been investigated (Chen, F., et al., Eur Neuropsychopharmacol. 2005 March; 15(2):193-8). The study suggested that paroxetine, although to a lesser extent than escitalopram, stabilized the ³H-paroxetine:human serotonin transporter complex at the primary high-affinity site. Sertraline, fluoxetine, venlafaxine, and duloxetine had little or no stabilizing effect on their binding to the primary binding site on the serotonin transporter (Chen, F., et al., Eur Neuropsychopharmacol. 2005 March; 15(2):193-8).

Whether a compound operates through an allosteric mechanism can be determined by in vitro dissociation experiments. Dissociation binding experiments measure the “off rate” (k_{off}) for a radioligand of the protein. After radioligand and transporter protein are allowed to bind (i.e. form a complex), then ligand is added to block further binding of radioligand to the transporter so that the rate of dissociation can be measured. Binding (as measured by radioactivity of the radioligand:transporter complex) is measured at various times to determine the rate at which the radioligand dissociates from the transporter. Dissociation rate constants can be used to determine the half-life of the bound complex. Half-life determinations can be used to ascertain whether a compound is an allosteric modulator of the human SERT.

Those of ordinary skill in the art can determine whether a compound, particularly an SRI, is an allosteric modulator of the human serotonin transporter (hSERT) as recited in the claims of this application, by determining the Z-factor for a compound by the method described in the following paragraphs.

To first determine the dissociation rate, isolated membranes from COS-1 cells transiently transfected with hSERT (GenBank Accession. No. X70697) are prepared by standard methods. Methods of transfection are also well known in the art. Hereinafter, assays are carried out in duplicate from at least three independent transfections using the same transfection method.

Initially, a radioligand/hSERT complex is formed during a 30-minute incubation of membrane preparations expressing hSERT and radioligand (radiolabeled-test compound) at 4° C. in buffer (50 mM Tris, pH 7.4; 120 mM NaCl, 5 mM KCl). Radioligand is present at a concentration approximately 10 times the K_{d} value for the radioligand. (K_{d} values are previously determined in the same buffer).

The radioligand/hSERT complex is diluted by 30-fold in the same buffer. In separate experiments the radioligand/hSERT complex is diluted by 30-fold in the same buffer containing test compound (cold, non-radiolabeled). Incubation of the radioligand/hSERT complex diluted in buffer with or without test compound continues for increasing time intervals at 20° C. At each time interval (e.g. 10 min., 20 min., 30 min., etc.), samples are removed from the incubation and the reaction is stopped by filtration through GF/C glass-fiber filters on a cell harvester. Accumulated radioactivity for each sample is determined by direct counting of plates using a Packard Bell microplate scintillation counter. The radioactivity represents binding and is expressed as fmol complex/mg membranes. Binding for each sample is plotted against increasing time to determine dissociation rate. The dissociation rate of the radioligand (k_{off}) is determined by non-linear regression using a GraphPad PRISM program (GraphPad Software, San Diego, Calif.). Dissociation half-life (t_{1/2}) is calculated by 0.69302/k_{off} and is represented in units of time.

Dissociation half-life of radioligand/hSERT complex (expressed in minutes) is plotted against increasing concentration of test compound in dissociation buffer (e.g. 10 μM, 20 μM, 30 μM, 40 μM, and 50 μM of test compound). The slope of this plot is termed a Z-factor. The Z-factor is calculated from at least four independent determinations. Z-factor is a measure of the degree of stabilization of the radioligand/hSERT complex. A Z-factor greater than 0 (zero) is indicative of a positive allosteric modulator. Thus, as used in the specification and the claims, an allosteric modulator is defined as a compound that has a Z-factor greater than 0 (zero) as determined by the above test.

Compounds of the invention that 1) bind to the primary binding site of the serotonin transporter having an IC₅₀ value of less than about 50 nM, and 2) bind to an allosteric site of the serotonin transporter having a Z-factor greater than zero (0), are considered to be serotonin dual action compounds. These compounds bind to the primary binding site of the serotonin transporter with high affinity, i.e. have stronger uptake inhibitory potency, and also bind to an allosteric site of the serotonin transporter to stabilize, or modulate the binding of ligands at the primary site to further enhance inhibition of 5-HT reuptake. Serotonin dual action compounds are also known as allosteric serotonin reuptake inhibitors, or ASRIs.

In still further embodiments, the serotonin dual action compound further binds to the primary binding site of the serotonin transporter having an IC₅₀ value of less than about 10 nM.

By way of non-limiting example to further illustrate the above definition of serotonin dual action compound, R-citalopram does not fall within the definition of serotonin dual action compound because R-citalopram binds to the primary binding site of the serotonin transporter having a reported IC₅₀ value of greater than 50 nM. See, for example, Sanchez, C. et al. Psychopharmacology 2003; 167:353-362.

Results and Discussion

In the mouse forced swim test, it was unexpected that the effects of SRIs in the mouse forced swim test were potentiated by co-administration of 5-HTP, as reflected by a change in ED₅₀-values for the SRI alone and in combination with 5-HTP in Table 1. The potentiation effect of 5-HTP, expressed as the ratio between ED₅₀-value for SRI alone and SRI in combination with 5-HTP, was more marked for the allosteric modulators escitalopram and paroxetine than for other SRIs (right column in Table 1).

TABLE 1
Effect of serotonin reuptake inhibitors (SRIs) in the mouse forced
swim test alone and in combination with 5-HTP (25 mg/kg, SC).

ED₅₀ (mg/kg)

| | | SRI + 5-HTP | |
|---|---|---|---|
| | SRI | (25 mg/kg) | Potentiation |
| Escitalopram | 12 | 0.42 | 29 |
| Paroxetine | 6.5 | 0.64 | 10 |
| Fluoxetine | >8.9 | 5.4 | >1.6 |
| Venlafaxine | >10 | 3.9 | >2.6 |

The minimal effective dose of 5-HTP in the mouse forced swim test was 10 mg/kg and the maximum potentiation effect was achieved at 50 mg/kg 5-HTP in combination with escitalopram at a dose that corresponds to a clinically effective concentration.

In the mouse forced swim test, co-administration of 5-HTP at doses of 10, 15, and 50 mg/kg significantly potentiates the response to escitalopram at a dose (0.5 mg/kg) that produces clinically relevant plasma levels (Sanchez, C. and Kreilgaard, M., Pharmacol Biochem Behav. 2004 February; 77(2):391-8). Doses of 25 and 50 mg/kg of 5-HTP, which are not by themselves effective in the mouse forced swim test, correspond to mouse plasma levels of 17 and 41 ng/ml, respectively (Magnussen, I., Acta Pharmacol Toxicol (Copenh). 1984 September; 55(3):199-202). In humans, 5-HTP has been shown to be effective in ameliorating the symptoms of depression (for review, see Birdsall, T. C., Altern Med Rev. 1998 August; 3(4):271-80). For this indication, typical doses of 100-200 mg of 5-HTP result in plasma levels of 50-100 ng/ml (Gijsman, H. J., et al., J Clin Psychopharmacol. 2002 April, 22(2):183-9). Thus, significant potentiating effects of 5-HTP on the efficacy of escitalopram in the mouse forced swim test are achieved at plasma levels that are at least 3 times lower than those required to achieve clinical efficacy in humans. Therefore, 5-HTP doses of approximately 34 mg (30-35 mg) given to a human may achieve plasma levels of approximately 17 ng/ml and thus still potentiate escitalopram. Furthermore, 5-HTP doses even 2.5 times lower, or at doses of approximately 13 mg (10-15 mg) may still achieve a strong potentiating effect of escitalopram.

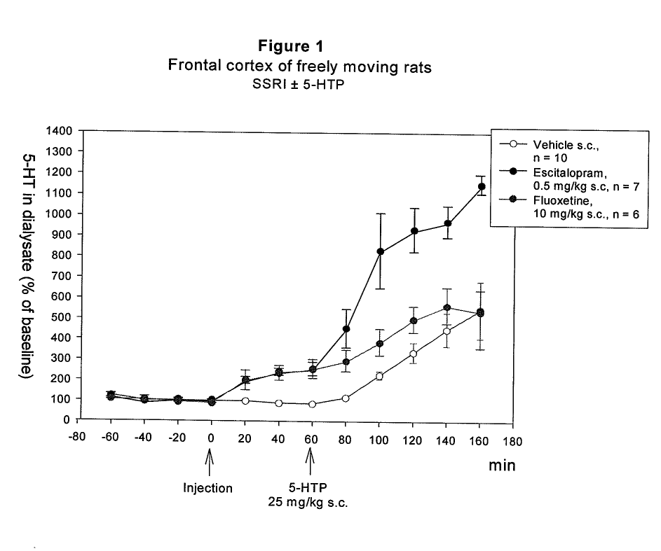

The larger 5-HTP potentiation effect of an allosteric modulator compared to a non-allosteric SRI is confirmed at the mechanistic level in the rat microdialysis model. The 5-HTP potentiation effect measured as increase of extra-cellular 5-HT in the frontal cortex is dramatically higher with the allosteric modulator, escitalopram, than with fluoxetine (FIG. 1), which is not considered to be an allosteric modulator.

In the mouse marble burying assay, escitalopram, administered without 5-HTP at a dose of 0.25 mg/kg IP, is inactive. However, when 5-HTP 2.5 mg/kg is administered to mice treated with escitalopram, 0.0625-0.25 mg/kg, a significant reduction in marble burying was observed. There is no behavioral effect of 5-HTP alone at this dose. The plasma levels achieved at the dose range of 0.0625-0.25 mg/kg escitalopram correspond to plasma levels and transporter occupancy well below those necessary to achieve clinical efficacy (Sanchez, C. and Kreilgaard, M., Pharmacol Biochem Behav. 2004 February; 77(2):391-8; Larsen, A. K. et al., Br J. Pharm. 2004, 141:1015-23). The clinically used dose range for escitalopram, which corresponds to approximately 70% receptor occupancy (Klein, N. et al, Eur Neuropsychopharmacol 2005, 15 (Suppl 3): S387), is 5-20 mg escitalopram. Thus a significant synergistic effect is achieved between escitalopram and 5-HTP even at doses that are below clinically used doses of both compounds.

What is claimed:

1. A pharmaceutical composition comprising (i) 5-hydroxytryptophan in an amount ranging from about 1 mg to about 75 mg; and (ii) a serotonin reuptake inhibitor.

2. The composition of claim 1, wherein the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

3. The composition of claim 1, wherein the serotonin reuptake inhibitor is a selective serotonin reuptake inhibitor.

4. The composition of claim 1, wherein the serotonin reuptake inhibitor is a serotonin dual action compound.

5. The composition of claim 1, wherein the serotonin reuptake inhibitor is escitalopram.

6. The composition of claim 5, wherein the composition comprises 5 mg to 30 mg of escitalopram.

7. The composition of claim 6, wherein the composition comprises 3 mg to 50 mg of 5-hydroxytryptophan.

8. The composition of claim 6, wherein the composition comprises 10 mg to 50 mg of 5-hydroxytryptophan.

9. The composition claim 1, wherein the serotonin reuptake inhibitor is paroxetine.

10. The composition of claim 9, wherein the composition comprises 10 mg to 60 mg paroxetine.

11. The composition of claim 10, wherein the composition comprises 3 mg to 50 mg of 5-hydroxytryptophan.

12. The composition of claim 10, wherein the composition comprises 10 mg to 50 mg of 5-hydroxytryptophan.

13. The composition of claim 1, further comprising a peripheral decarboxylation inhibitor.

14. The composition of claim 13, wherein the peripheral decarboxylation inhibitor is carbidopa.

15. The composition of claim 14, wherein said composition contains carbidopa in an amount ranging from about 100 mg to about 150 mg.

16. The composition of claim 15, wherein said composition is a tablet or a capsule.

17. A pharmaceutical composition comprising (i) a subclinical dose of a serotonin reuptake inhibitor; and (ii) 5-hydroxytryptophan.

18. The composition of claim 17, wherein the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

19. The composition of claim 17, wherein the serotonin reuptake inhibitor is a selective serotonin reuptake inhibitor.

20. The composition of claim 17, wherein the serotonin reuptake inhibitor is a serotonin dual action compound.

21. The composition of claim 20, wherein said composition contains 5-hydroxytryptophan in an amount ranging from about 1 mg to about 600 mg.

22. The composition of claim 20, wherein said composition contains 5-hydroxytryptophan in an amount ranging from about 25 mg to about 300 mg.

23. The composition of claim 20, wherein said composition contains 5-hydroxytryptophan in an amount ranging from about 50 mg to about 200 mg.

24. The composition of claim 17, wherein said composition comprises (i) escitalopram in an amount less than 5 mg and (ii) 5-hydroxytryptophan in an amount ranging from about 1 mg to about 600 mg.

25. The composition of claim 17, wherein said composition comprises (i) escitalopram in an amount less than 5 mg and (ii) 5-hydroxytryptophan in an amount ranging from about 25 mg to about 300 mg.

26. The composition of claim 17, wherein said composition comprises (i) escitalopram in an amount less than 5 mg and (ii) 5-hydroxytryptophan in an amount ranging from about 50 mg to about 200 mg.

27. The composition of claim 17, wherein said composition comprises (i) paroxetine in an amount less than 10 mg and (ii) 5-hydroxytryptophan in an amount ranging from about 1 mg to about 600 mg.

28. The composition of claim 17, wherein said composition comprises (i) paroxetine in an amount less than 10 mg and (ii) 5-hydroxytryptophan in an amount ranging from about 25 mg to about 300 mg.

29. The composition of claim 17, wherein said composition comprises (i) paroxetine in an amount less than 10 mg and (ii) 5-hydroxytryptophan in an amount ranging from about 50 mg to about 200 mg.

30. The composition of claim 17, further comprising a peripheral decarboxylation inhibitor.

31. The composition of claim 30, wherein the peripheral decarboxylation inhibitor is carbidopa.

32. The composition of claim 31, wherein said composition contains carbidopa in an amount ranging from about 100 mg to about 150 mg.

33. The composition of claim 32, wherein said composition is a tablet or a capsule.

34. A pharmaceutical composition comprising (i) a serotonin reuptake inhibitor; and (ii) 5-hydroxytryptophan.

35. The composition of claim 34, wherein the serotonin reuptake inhibitor is selected from citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

36. The composition of claim 34, wherein the serotonin reuptake inhibitor is a selective serotonin reuptake inhibitor.

37. The composition of claim 34, wherein the serotonin reuptake inhibitor is a serotonin dual action compound.

38. The composition of claim 37, wherein said composition contains 5-hydroxytryptophan in an amount ranging from about 1 mg to about 600 mg.

39. The composition of claim 37, wherein said composition contains 5-hydroxytryptophan in an amount ranging from about 25 mg to about 300 mg.

40. The composition of claim 37, wherein said composition contains 5-hydroxytryptophan in an amount ranging from about 50 mg to about 200 mg.

41. The composition of claim 34, wherein said composition comprises (i) escitalopram in an amount from about 5 mg to about 30 mg and (ii) 5-hydroxytryptophan in an amount ranging from about 1 mg to about 600 mg.

42. The composition of claim 34, wherein said composition comprises (i) escitalopram in an amount from about 5 mg to about 30 mg and (ii) 5-hydroxytryptophan in an amount ranging from about 25 mg to about 300 mg.

43. The composition of claim 34, wherein said composition comprises (i) escitalopram in amount from about 5 mg to about 30 mg and (ii) 5-hydroxytryptophan in an amount ranging from about 50 mg to about 200 mg.

44. The composition of claim 34, wherein said composition comprises (i) paroxetine in an amount from about 10 mg to about 60 mg and (ii) 5-hydroxytryptophan in an amount ranging from about 1 mg to about 600 mg.

45. The composition of claim 34, wherein said composition comprises (i) paroxetine in an amount from about 10 mg to about 60 mg and (ii) 5-hydroxytryptophan in an amount ranging from about 25 mg to about 300 mg.

46. The composition of claim 34, wherein said composition comprises (i) paroxetine in an amount from about 10 mg to about 60 mg and (ii) 5-hydroxytryptophan in an amount ranging from about 50 mg to about 200 mg.

47. The composition of claim 34, further comprising a peripheral decarboxylation inhibitor.

48. The composition of claim 47, wherein the peripheral decarboxylation inhibitor is carbidopa.

49. The composition of claim 48, wherein said composition contains carbidopa in an amount ranging from about 100 mg to about 150 mg.

50. The composition of claim 49, wherein said composition is a tablet or a capsule.

51. The composition of claim 50, wherein said composition is in a unitary dosage form.
